(19) United States
(12) Patent Application Publication
(10) Pub. No.: US 2010/0233824 A1
(43) Pub. Date: Sep. 16, 2010

(54) MICROFLUIDIC METHODS AND SYSTEMS FOR USE IN DETECTING ANALYTES

(75) Inventors: Godefridus Johannes Verhoeckx, Eindhoven (NL); Henkrik Sibolt Van Damme, Eindhoven (NL); Jeroen Hans Nieuwenhuis, Eindhoven (NL); Sergei Shulepov, Eindhoven (NL); Johannes Wilhelmus Weekamp, Eindhoven (NL); Mara Johanna Jacoba Sijbers, Eindhoven (NL); Harold Johannes Antonius Brans, Eindhoven (NL); Femke Karina De Theije, Eindhoven (NL); Albert Hendrik Jan Immink, Eindhoven (NL)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., EINDHOVEN (NL)

(21) Appl. No.: 12/669,213

(22) PCT Filed: Jul. 11, 2008
(86) PCT No.: PCT/IB2008/052803

§ 371 Date: Jun. 1, 2010

(30) Foreign Application Priority Data
Jul. 20, 2007 (EP) 07112834.2

(51) Int. Cl. G01N 33/53; G01N 33/00; B23P 17/04

(52) U.S. Cl. 436/501; 422/68.1; 29/428

(57) ABSTRACT

A microfluidic reactor arrangement (100) for use in detecting an analyte in a fluid sample (106) is described. The reactor arrangement is provided with a reagent providing means such that the reagent can be introduced after assembly of the reactor arrangement. The latter can be performed by introducing the reagent in the form of a solution or a dispersion and fixing it on a holding means (118) by removal of the liquid, i.e. by drying, the holding means comprising the reagent in a solid version in the detector chamber. Prior to the introduction of the reagent, components of the reactor arrangement already present, such as the sample inlet can be hydrophilised by a wetting hydrophilising technique. The invention relates to a manufacturing technique as well as to the resulting product. The invention furthermore relates to functionalizing of the reactor arrangement with a particular reagent for particular applications. The latter can be performed well after fabrication and assembly of the major reactor arrangement components.

### FIELD OF THE INVENTION

The present invention relates to the field of (bio)reactors, such as for example (bio)sensors. More particularly, the present invention relates to methods and systems for obtaining micro fluidic devices for use in detecting the presence of an analyte, e.g., for qualitative or quantitative detection of biological, chemical or biochemical entities.

### BACKGROUND OF THE INVENTION

(Bio)reactors are devices that allow the contacting of various reagents in a controlled manner in order to obtain a product. By using (bio)reactors, factors such as the quantity of reagent, the temperature, duration, physico-chemical characteristics, sequence etc. . . . of the reaction to be performed, can be controlled. (Bio)reactors can be destined to multiple or single use. Amongst (bio)reactors, biosensors are devices that allow qualitative or quantitative detection of target molecules, also called “analytes”, such as, e.g., proteins, viruses, bacteria, sperm/semen, cells, cell components, cell membranes, spores, DNA, RNA, etc. . . . in a sample fluid comprising for example blood, serum, plasma, saliva, tissue extract, intestinal fluid, cell culture extract, food or feed extract, drinking water, etc. Often a biosensor uses a sensor surface that comprises specific recognition elements for capturing the analyte. The surface of the biosensor device may therefore be modified by attaching specific molecules to it, which are suitable to bind the target molecules to be detected in the sample fluid. A well-established principle is the counting of labeled molecules of interest captured at predetermined sites on the biosensor. For example, such molecules of interest may be labeled with magnetic particles or beads and these magnetic particles or beads can be detected with a magnetic sensor. One possible alternative is the detection of the amount of analyte using optical detection such as fluorescence. In this case, the analyte itself may carry a fluorescent label, or alternatively an additional incubation with a fluorescent-labeled recognition element may be performed.

In most biosensors, the sensor device is provided with a dry reagent in addition to the sensor surface. The reagent may comprise, e.g., labels coupled to biologically active moieties, e.g., an anti-drug antibody. In order to limit the analysis time, the reagent can be deposited directly on the sensor surface. When the fluid sample arrives, the dry reagent dissolves and mixes into the fluid, which then wets the sensor surface. The labels, as well as the sensor surface, are exposed to the target molecules (e.g., drug). This influences the binding of the labels onto the sensor surface, which is detected. An inconvenience of having the reagent deposited directly on the sensor surface is that it leads to possible premature reaction of the reagent with the sensor surface (i.e., before the reagent has had the possibility to react with the target), thus disturbing the detection.

A device suitable for detecting the presence of an analyte in a sample fluid is known from U.S. Application No. 2004/0115094. In this patent application, the device comprises a first body comprising a sensor module and a fluidic system. This first body is connected to a second body provided with an inlet and an outlet for the sample fluid, and a channel connecting the inlet and the outlet. The device is formed by assembling the first and second bodies. By doing so, the fluidic system and the sensor connect to one another in a suitable manner for the transport of fluid. From the construction of such a device, it appears that the introduction of a reagent can only be done in the device before the assembly of the two bodies. In many biosensors it is advantageous to let the device fill with sample fluid with minimal interference of the user, i.e., let it fill autonomously. This can be achieved by letting the device fill by capillary forces. For this devices with hydrophilic walls are used. It is therefore advantageous to coat the various parts that will be in contact with the sample fluid with a hydrophilic material (e.g., adsorbing surfactants or hydrophilic polymers). This is most adequately performed by flushing the assembled device with a hydrophilic coating solution. Such a coating enables/facilitates the filling of the device with the sample fluid by autonomous flow. In many cases, a gluing process is not possible or efficient once the parts are coated. As a consequence, the coating process is typically carried out after gluing both parts of the cartridge together. To this end the assembled device is generally flushed with a solution of a suitable hydrophilisation agent. This procedure can obviously not be carried out when a reagent is in the device since the reagent would be dispersed in the hydrophilisation solution and washed away. Additionally, it appears from this construct that the solvent and samples are fed from the same opening, leading to a potential dilution of the samples or an improper homogenization with the reagent. There is therefore a need in the art for new improved devices and methods for detecting the presence of an analyte in a sample fluid.

### SUMMARY OF THE INVENTION

It is an object of the present invention to provide good systems, devices and methods for use in allowing an interaction with a sample fluid, as well as manufacturing methods for such devices and systems. It is an advantage of embodiments according to the present invention that good systems, devices and methods are provided for use in detecting an analyte in a sample fluid, as well as manufacturing methods for such devices and systems. It is an advantage of embodiments of the present invention to provide devices enabling the provision of a reagent in the device after its assembly, e.g. directly before the provision of the sample fluid in the device. It is also an advantage of embodiments according to the present invention to enable the contact between the sample fluid and the reagent before any contact between the reagent and the sensor occurs. Advantages of embodiments of the present invention include, but are not limited to, reliability and reproducibility of measurements and ease of manufacturing of the devices as well as reduction in lowering value in warehoused products. Another advantage of embodiments of the present invention is that (part of the) customization of the device takes place relatively late in the production process, which may be advantageous when a family of different products is made based on the same device but using different types or amounts of reagents, i.e. it allows for a late functionalisation and/or customization of the device after it has been assembled. It is an advantage of certain embodiments of the present invention to allow for the control of the incubation period and temperature when the sample fluid and the reagent are in contact.

The above objective is accomplished by a method and device according to the present invention.

A first aspect of the invention provides microfluidic reactor arrangement, the reactor arrangement comprising a housing having an outer wall enclosing a reaction chamber, the reaction chamber having an interaction surface, the outer wall having at least one sample inlet for introduction of the fluid sample and at least one reagent providing means distinct from the sample inlet for introducing at least one reagent into the reaction chamber thus providing said reagent on at least one holding means for holding a solid version of the at least one reagent at a reagent region within the reaction chamber, said holding means being located or locatable on a selected surface distinct from the interaction surface within the reaction chamber so that the reagent held by the holding means comes into fluid contact with the interaction surface when the fluid sample is introduced into the reaction chamber.

The microcluidic reactor arrangement may be a microfluidic sensor arrangement for use in detecting an analyte in a fluid sample, whereby the sensor arrangement comprises a housing having an outer wall enclosing a detection chamber, the detection chamber having a sensing surface, the outer wall having: at least one sample inlet for introduction of the fluid sample, and at least one reagent providing means distinct from the sample inlet for introducing at least one reagent into the detection chamber thus providing the reagent on at least one holding means for holding a solid version of the at least one reagent at a reagent region within the detection chamber, the holding means being located or locatable on a selected surface distinct from the sensor surface within the detection chamber so that the reagent held by the holding means comes into fluid contact with the sensing surface when the fluid sample is introduced into the detection chamber. It is an advantage of some embodiments according to the present invention that the reagent can be introduced in the sensor arrangement after wet hydrophilisation of the sample inlet. It is a further advantage of some embodiments according to the present invention that the reagent can be introduced in the sensor arrangement and held as a solid, e.g. freeze dried, manner on a selected position in the detection chamber while still allowing an efficient hydrophilisation of the sample inlet. This has the advantage that the sensor arrangement can be easily stored and that the amount of reagent can be accurately controlled.

In a particular embodiment of the microfluidic sensor arrangement of the invention, the reagent providing means may comprise a microfluidic transport means for delivering fluid reagent to the at least one holding means. It is an advantage of embodiments according to the present invention that the reagent can be introduced in the detection chamber in a liquid form. It is furthermore an advantage of embodiments according to the present invention that good metering of the amount of reagent can be obtained. In a further particular embodiment of the micro fluidic sensor arrangement of the invention, the holding means may be a separate cover connectable to the outer wall of the microfluidic sensor arrangement. It is an advantage of embodiments according to the present invention that functionalising of the sensor arrangement can be performed late in the manufacturing process. The separate cover may be connected by gluing, screwing, clipping, clicking and the like.

In another embodiment of the invention, the holding means of the sensor arrangement of the invention may be adapted for comprising a predetermined amount of reagent. More particularly, the holding means of the sensor arrangement of the invention may comprise an open capillary channel. It is an advantage of embodiments according to the present invention that the amount of reagent provided on the holding means can be accurately determined, e.g. by the length and size of the open capillary channel used.

In further particular embodiments, the sensor arrangement of the invention may comprise a plurality of reagent providing means, each of the plurality of reagent providing means being adapted for delivering a reagent.

In yet another embodiment of the invention, the sample inlet may be hydrophilic. It is an advantage of embodiments according to the present invention that multiplexing may be performed, resulting in the possibility to accurately assess the presence and/or quantity of a plurality of analytes in the sample. It is an advantage of embodiments according to the present invention that filling of the cartridge with sample by autonomous flow can be obtained using a hydrophilic sample inlet. The microfluidic transport means, the holding means and/or the reagent inlet may be hydrophilic.

Moreover, in further embodiments, the reagent providing means of the sensor arrangement of the invention may comprise a capillary. It is an advantage of embodiments according to the present invention that the reagent may be provided using capillary forces, thus avoiding the need to a separate pumping means.

In particular embodiment of the invention, the sensor arrangement may further comprise a sample outlet for removing the fluid sample from the detection chamber, the sample outlet being distinct from the sample inlet and the reagent providing means.

In yet other embodiments of the invention, the holding means of the sensor arrangement of the invention may be connected to a reagent overflow chamber.

It is an advantage of embodiments according to the present invention that the amount of reagent provided in the detection chamber can be accurately selected, whereby excess of reagent is collected in a reagent overflow chamber. The overflow chamber may comprise a capillary. The reagent overflow chamber may be hydrophilic.

In alternative embodiments of the invention, the sensor arrangement may comprise an excess reagent detection means for detecting excess liquid reagent. It is an advantage of embodiments according to the present invention that the sensor arrangement may comprise a metering system for determining the amount of reagent to be provided and to check appropriate loading of the holding means.

In yet other embodiments of the invention, the microfluidic sensor arrangement may comprise at least one reagent in a solid version in the holding means.

A second aspect of the invention provides a microfluidic reactor arrangement for use in detecting an analyte in a fluid sample, the reactor arrangement comprising a housing having an outer wall enclosing a reaction chamber, the outer wall having at least one sample inlet covered with a hydrophilic coating, the sample inlet for introduction of the fluid sample and the reaction chamber having an interaction surface and the outer wall having at least one holding means comprising a solid version of at least one reagent at a reagent region within the reaction chamber, said holding means being located on a selected surface within the reaction chamber so that the solid reagent held by the holding means comes into fluid contact with the interaction surface when the fluid sample is introduced into the reaction chamber. The micro fluidic reactor arrangement may be a microfluidic sensor arrangement for use in detecting an analyte in a fluid sample, the sensor arrangement comprising a housing having an outer wall enclosing a detection chamber, the outer wall having at least one sample inlet covered with a hydrophilic coating, the sample inlet for introduction of the fluid sample; the detection chamber having a sensing surface and the outer wall having at least one holding means comprising a solid version of at least one reagent at a reagent region within the detection chamber, the holding means being located or locatable on a selected surface within the detection chamber so that the solid reagent held by the holding means comes into fluid contact with the sensing surface when the fluid sample is introduced into the detection chamber.

Further embodiments of this second aspect of the invention provides a microfluidic sensor arrangement that may comprise a micro fluidic transport means separate from the sample inlet for providing reagent to the holding means.

In yet another embodiment, the holding means of the microfluidic sensor arrangement of the invention may comprise an open channel for holding the solid reagent.

A third aspect of the invention provides a method for manufacturing a microfluidic reaction arrangement, the method comprising the step of providing an interaction surface, providing a housing enclosing an interaction surface and forming a reaction chamber, the providing a housing comprising providing a housing with a sample inlet and at least one reagent providing means, distinct from the sample inlet, for introducing at least one reagent into the reaction chamber by providing the reagent on at least one holding means distinct from the interaction surface for holding a solid version of at least one reagent at a reagent region within the reaction chamber, the holding means being positioned on a selected surface within the reaction chamber so that the reagent held by the holding means comes into fluid contact with the interaction surface when the fluid sample is introduced in the reaction chamber. The micro fluidic reactor arrangement may be a micro fluidic sensor arrangement whereby the reaction chamber may be a detection chamber and the interaction surface may be a sensing surface.

Particular embodiments of this third aspect of the invention encompasses methods further comprising hydrophilising the sample inlet by introducing a hydrophilisation liquid in the detection chamber through the sample inlet after the providing a housing and prior to introducing reagent in the sensor arrangement. Hydrophilising of the sample inlet therefore can be done prior to the introduction of reagent.

Further embodiments of the invention may further envision providing a reagent overflow chamber connected to the at least one holding means, which may comprise excess detection means for detecting excess reagent liquid in said overflow chamber. It is an advantage of embodiments according to the present invention that a predetermined amount of reagent can be provided on the holding means. It is an advantage of embodiments according to the present invention that control and/or correction mechanisms can be provided for determining whether the predetermined amount of reagent is provided on the holding means.

In yet other embodiments of the invention, the methods may comprise introducing a predetermined amount of the at least one reagent via a micro fluidic transport means into the holding means and obtaining a solid version of the reagent thereon.

A fourth aspect of the invention provides methods for functionalising at least one microfluidic reactor arrangement comprising a reaction chamber enclosed by an outer wall, the outer wall having a sample inlet and a reagent providing means, the method comprising introducing a predetermined amount of at least one reagent into the reaction chamber via the reagent providing means distinct from the sample inlet thus providing the reagent on at least one holding means distinct from the interaction surface in the reaction chamber and holding on the at least one holding means a solid version of the predetermined amount of the at least one reagent at a reagent region within the reaction chamber at a selected surface within the reaction chamber so that the reagent held comes into fluid contact with the interaction surface when the fluid sample is introduced in the reaction chamber. The microfluidic reactor arrangement may be a microfluidic sensor arrangement whereby the reaction chamber may be a detection chamber and the interaction surface may be a sensing surface.

In certain embodiments of this fourth aspect of the invention, the methods may comprise detecting an excess of the reagent for controlling the amount of reagent provided on the holding means.

In another embodiment of the invention, the method may comprise, prior to the introducing, selecting a reagent from a plurality of reagents.

In a fifth aspect of the invention, methods are provided for detecting an analyte in a fluid sample comprising the step of introducing, via a sample inlet and based on hydrophilic forces, a fluid sample into a microfluidic sensor arrangement, the microfluidic sensor arrangement comprising a detection chamber, the detection chamber comprising a sensing surface and a predetermined amount of reagent in a solid form, the method further comprising contacting the fluid sample with the predetermined amount of reagent, thereby forming a fluid mixture, the reagent being accessible to the fluid sample from within the detection chamber; contacting the fluid mixture with the sensing surface; and detecting an interaction between the fluid mixture and the sensing surface.

In a sixth aspect of the invention provides a use of a microfluidic sensor arrangement for detecting an analyte in a fluid sample.

Particular and preferred aspects of the invention are set out in the accompanying independent and dependent claims. Features from the dependent claims may be combined with features of the independent claims and with features of other dependent claims as appropriate and not merely as explicitly set out in the claims.

The teachings of the present invention permit the design of improved methods and apparatuses for use in detecting analytes in a sample fluid.

The above and other characteristics, features and advantages of the present invention will become apparent from the following detailed description, taken in conjunction with the accompanying drawings, which illustrate, by way of example, the principles of the invention. This description is given for the sake of example only, without limiting the scope of the invention. The reference figures quoted below refer to the attached drawings.

### BRIEF DESCRIPTION OF THE DRAWINGS

FIG. 1 is a schematic representation of a micro fluidic sensor arrangement for sensing or detecting at least one analyte in a sample according to an embodiment of the present invention.

FIG. 2 is a vertical cross-section of a micro fluidic sensor arrangement comprising a reagent inlet for providing reagent to a holding means for holding a solid form of the reagent according to an embodiment of the present invention.

FIG. 3 illustrates a schematic representation of a capillary in a holding means of a microfluidic sensor arrangement, according to an embodiment of the present invention.

FIG. 4 is a vertical cross-section of a micro fluidic sensor arrangement comprising a reagent outlet and a sensing means for controlling the amount of reagent provided, according to an embodiment of the present invention.

FIG. 5 is a vertical cross-section of a micro fluidic sensor arrangement comprising a detection chamber delimited by a connectable cover at its top side, according to an embodiment of the present invention.

FIG. 6 is a detailed vertical cross-section of the sensor arrangement as shown in FIG. 5 without the cover.

FIG. 7 is a detailed vertical cross-section of the cover carrying the reagents as shown in FIG. 5.

In the different figures, the same reference signs refer to the same or analogous elements.

### DETAILED DESCRIPTION OF THE EMBODIMENTS

The present invention will be described with respect to particular embodiments and with reference to certain drawings but the invention is not limited thereto but only limited by the claims. Any reference signs in the claims shall not be construed as limiting the scope. The drawings described are only schematic and are non-limiting. In the drawings, the size of some of the elements may be exaggerated and not drawn on scale for illustrative purposes.

Where the term “comprising” is used in the present description and claims, it does not exclude other elements or steps. Where an indefinite or definite article is used when referring to a singular noun e.g. “a” or “an”, “the”, this includes a plural of that noun unless something else is specifically stated.

Furthermore, the terms first, second, third and the like in the description and in the claims, are used for distinguishing between similar elements and not necessarily for describing a sequence, either temporally, spatially, in ranking or in any other manner. It is to be understood that the terms so used are interchangeable under appropriate circumstances and that the embodiments of the invention described herein are capable of operation in other sequences than described or illustrated herein.

Moreover, the terms top, bottom, over, under, vertical and the like in the description and the claims are used for descriptive purposes and not necessarily for describing relative positions. It is to be understood that the terms so used are interchangeable under appropriate circumstances and that the embodiments of the invention described herein are capable of operation in other orientations than described or illustrated herein.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may do so. Furthermore, the particular features, structures or characteristics may be combined in any suitable manner, as would be apparent to one of ordinary skill in the art from this disclosure, in one or more embodiments.

Similarly it should be appreciated that in the description of exemplary embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the detailed description are hereby expressly incorporated into this detailed description, with each claim standing on its own as a separate embodiment of this invention.

Furthermore, while some embodiments described herein include some but not other features included in other embodiments, combinations of features of different embodiments are meant to be within the scope of the invention, and form different embodiments, as would be understood by those in the art. For example, in the following claims, any of the claimed embodiments can be used in any combination.

Furthermore, an element described herein of an apparatus embodiment is an example of a means for carrying out the function performed by the element for the purpose of carrying out the invention.

In the description provided herein, numerous specific details are set forth. However, it is understood that embodiments of the invention may be practiced without these specific details. In other instances, well-known methods, structures and techniques have not been shown in detail in order not to obscure an understanding of this description.

The following terms or definitions are provided solely to aid in the understanding of the invention. The definitions should not be construed to have a scope less than understood by a person of ordinary skill in the art.

The term “coupled” when used herein and unless specified otherwise, should not be interpreted as being restricted to direct connections only. The terms “coupled” and “connected”, along with their derivatives, may be used. It should be understood that these terms are not intended as synonyms for each other. Thus, the scope of the expression “a device A coupled to a device B” should not be limited to devices or systems wherein an output of device A is directly connected to an input of device B. It means that there exists a path between an output of A and an input of B which may be a path including other devices or means. “Coupled” may mean that two or more elements are either in direct physical contact, or that two or more elements are not in direct contact with each other but yet still co-operate or interact with each other.

The term “sample”, as used herein, relates to a composition which may comprise at least one analyte of interest. The sample is preferably fluid, also referred to as “sample fluid”, e.g., an aqueous composition. The term “analyte” as used herein refers to a substance whose presence, absence, or concentration is to be determined by using embodiments of the present invention. Analytes may include, but are not limited to organic molecules, metabolites such as glucose or ethanol, proteins, peptides, nucleic acid segments, molecules such as pharmaceuticals, antibiotics or drugs, drugs of abuse, molecules with a regulatory effect in enzymatic processes such as promoters, activators, inhibitors, or cofactors, viruses, bacteria, cells, cell components, cell membranes, spores, DNA, RNA, micro-organisms and fragments and products thereof, or any substance for which attachment sites, binding members or receptors (such as antibodies) can be developed.

The term “label” as used herein refers to a molecule or material capable of generating a detectable signal or capable of binding to another molecule or forming a complex which generates a detectable signal. Suitable labels for use in different detection systems and methods of the present invention are numerous and extensively described in the art. These may be optical labels (e.g. luminescent molecules like fluorescent agents, phosphorescent agents, chemiluminescent agents, bioluminescent agents and the like-colored molecules, molecules producing colours upon reaction), radioactive labels, magnetic and/or electric labels, enzymes, specifically recognizable ligands, micro-bubbles detectable by sonic resonance and the like. Labels can be direct labels, which can be detected by a sensor. Alternatively, labels can be indirect labels, which become detectable after a subsequent development process. The label used in the methods of the present invention may be an analyte-specific label, i.e., capable of binding specifically to the analyte. Nevertheless, it is also envisaged that where the analyte is present in a purified form, it is sufficient that the label binds to the analyte.

The term “analyte analogue”, as used herein, refers to a substance that can associate with a probe or capture probe used for capturing or binding analytes. The analyte analogue is used in competitive assays where the analyte is determined based on competition with the analyte analogue, e.g., in the competitive binding to a probe or capture probe.

The term “probe” relates in the present invention to a binding molecule that specifically binds an analyte. Probes envisaged within the context of the present invention include biologically-active moieties such as, but not limited to, whole anti-bodies, antibody fragments such as Fab′ fragments, single chain Fv, single variable domains, VHH, heavy chain antibodies, peptides, epitopes, membrane receptors or any type of receptor or a portion thereof, substrate-trapping enzyme mutants, whole antigenic molecules (haptens) or antigenic fragments, oligopeptides, oligonucleotides, mimitopes, nucleic acids and/or mixture thereof, capable of selectively binding to a potential analyte. Antibodies can be raised to non-proteinaceous compounds as well as to proteins or peptides. Probes may be members of immunoreactive or affinity reactive members of binding-pairs. The nature of the probe will be determined by the nature of the analyte to be detected. Most commonly, the probe is developed based on a specific interaction with the analyte such as, but not limited to, antigen-antibody binding, complementary nucleotide sequences, carbohydrate-lectin, complementary peptide sequences, ligand-receptor, coenzyme, enzyme inhibitors-enzyme, etc. . . . In the present invention, the function of a probe is to specifically interact with an analyte to permit its detection. Therefore, probes may be labeled or may be directly or indirectly detectable. The probe can be an anti-analyte antibody if, for instance, the analyte is a protein.

Alternatively, the probe can be a complementary oligonucleotide sequence if, for instance, the analyte is a nucleotide sequence.

The term “capture probe” as used herein, refers to probes for immobilizing analytes and/or labeled analytes on a sensor surface via recognition or binding events.

The term “sensor” as used herein refers to a device allowing qualitative and/or quantitative detection of an analyte in a sample fluid. If the analyte is of biological nature or if the sensor relies on biological entities for detection, (e.g. antibodies capture probes) the sensor will sometimes be referred as a “biosensor”. The “sensor” as used herein usually operates its sensing through a sensing surface that will either capture analytes or exchange an analyte analogue immobilized thereon for an analyte present in the sample fluid.

Whereas in the following description features of aspects and embodiments of the present invention are set forth with respect to a micro fluidic sensor arrangement, the aspects and embodiments of the present invention also relate to a microfluidic reactor arrangement, wherein controlled reaction with a sample fluid can be obtained. The reactor may be a bioreactor. The reactor may be adapted for contacting various reagents in a controlled manner with a sample fluid in order to obtain a product. The reactor does not require the detector and sensing surface as described in the aspects and embodiments below. In the reactor, the sensing surface is replaced by an interaction surface, whereby e.g. a particular type of further reagent is provided. In embodiments of the present invention, the reactor thus may e.g. be adapted for providing a reagent after assembly of the arrangement such that the reagent does not interact, before contacting with the sample fluid, with other reagents provided at an interaction surface on a different place in the reaction chamber. Thus whereas the embodiments are described with respect to a sensor arrangement, the concepts provided can mutates mutandis be applied to a reactor with an interaction surface instead of a sensing surface, optionally provided with further reagents.

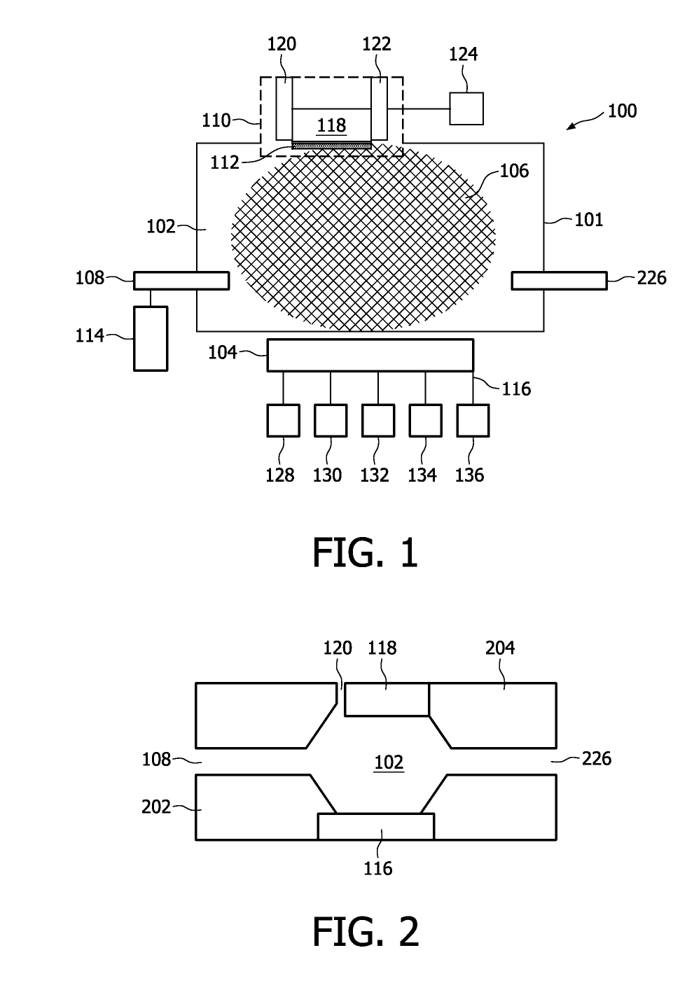

In a first aspect, the present invention relates to a microfluidic sensor arrangement for use in detecting the presence of an analyte in a sample fluid. The microfluidic sensor arrangement may be for example suitable for use in sensing applications for detecting biological, chemical or biochemical analytes in a fluid sample. The microfluidic sensor arrangement also may allow the contacting of various reagents in a controlled manner in order to obtain a product, e.g. it may be a reactor arrangement. A schematic representation of such a microfluidic sensor arrangement 100 is indicated in FIG. 1. The microfluidic sensor arrangement 100 comprises a housing having an outer wall 101 and a detection chamber 102, enclosed, or substantially enclosed by the outer wall 101, wherein the detection will occur. The detection chamber 102 enclosed by the outer wall may e.g. be formed by assembling a first part comprising a sensor surface and a second component comprising microfluidic parts, although the invention is not limited thereto. The detection chamber 102 is at least partially delimited by a sensor surface 104 that is accessible to the sample fluid 106, when introduced, from within the detection region. The detection chamber 102 may have a fixed volume, or optionally a volume that is first fixed after tuning or adapting this volume. The latter is advantageous, e.g. if a quantitative detection is required. In a detection chamber 102 with fixed volume, a fixed volume of fluid 106 can be provided. The volume of the detection chamber 102 may be any suitable volume for detection, e.g. but not limited to a volume comprised between 0.1 and 10 μl. A detection chamber 102 with well-defined volume also is preferred if a competitive assay is performed, as the sample volume is important and the concentration of labels determines the result. The number of labels can be defined by providing, e.g., dosing, a well-defined volume of a well-defined concentration of labels, in combination with a well-defined volume resulting in a correct number of labels per volume of sample fluid 106.

The outer wall 101 of the sensor device 100 comprises a sample inlet 108 for the fluid sample 106. The sample inlet 108 for the fluid sample 106 has an inlet opening in the detection chamber 102 distinct from a reagent providing means 110, e.g. an inlet opening of a reagent inlet, for introducing a reagent which will be held as a solid version 112 of the reagent in the detection chamber 102. The sample inlet 108 for the fluid sample may comprise a capillary conduit (herein referred to as “capillary”), e.g., a tube or a hollow section with dimensions such that liquid, e.g., a liquid fluid sample, can be driven therein via capillary forces. Typical dimensions for the diameter of capillary sections are 0.1 to 2 mm. Optionally, the device 100 may further or alternatively comprise pressure means 114 for forcing the fluid sample 106 through the sample inlet 108 for fluid sample. Suitable pressure means comprise but are not limited to, e.g., pumps, syringes and the likes. Such pressure may be provided in micro fluidic format as is known to the skilled person. The pressure means 114 may provide a positive pressure for forcing the fluid sample into the detection chamber 102, or it may create a vacuum or low pressure applied at the side of the detection chamber 102 of the device 100 for pulling the fluid sample in the detection chamber 102. The sample inlet 108 may be hydrophilised by wetting it with a hydrophilising liquid.

It is an advantage of embodiments according to the present invention that reagent providing means are provided that allow loading of the reagent after assembly of the major components of the sensor arrangement 100, e.g. including sensor surface, outer wall and sample inlet 108, and such that hydrophilising of the sample inlet 108 can be done prior to loading of a reagent, e.g. loading of a dissolvable reagent.

The sensor surface 104 may be constituted by the solid surface of the sensor 116 used. The sensor 116 may be part of the microfluidic sensor arrangement 100 or the sensor may be included in part at least in an external sensor that is part of a cartridge reader and the micro fluidic sensor arrangement 100 may be a cartridge that is suitable for introduction into the cartridge reader and for using the external sensor for obtaining a read-out. Also an external detector may be used, e.g. housed in the cartridge reader. The external detector is then used to detect changes on the sensor surface 104, e.g. optical variations that can be viewed by the detector through a window outer wall 101. The sensor surface 104 may comprise biologically or biochemically active moieties for capturing particles of interest. Biologically or biochemically active moieties may for example refer to capture probes and/or analyte analogs that are attached to the sensor surface and that are capable of binding, or that are reactive with, an analyte or labeled probe, respectively, when in appropriate conditions. The capture probes and/or analyte analogs of the biologically active layer may be retained or immobilized on the surface by any method known in the art. These biologically active moieties may be attached to the sensor surface 104 in a site-specific manner, meaning that the specific sites on these moieties are involved in the coupling, e.g., through a protein-resistant layer on the surface 104. The sensor surface 104 may have a porous surface in order to enhance the surface-over-volume ratio.

The outer wall 101 furthermore comprises at least one reagent providing means 110. The reagent providing means 110 has the advantage that it allows loading of the reagent after at least the sample inlet has been formed in the sensor arrangement allowing specific treatments of the sample inlet or other components prior to loading the reagent. The reagent providing means 110 is distinct from the sample inlet 108, it is a separate inlet at a separate location of the wall 101. It is adapted for introducing at least one reagent into the detection chamber and for providing the reagent on at least one holding means 118 for holding a solid version of the reagent at a reagent region within the detection chamber 102. The reagent providing means 110 may for example be adapted for introducing the reagent in a liquid or a solid version. As a solid the reagent may be introduced by introducing the holding means 118, e.g. covering a reagent inlet provided by the reagent providing means 110 by a holding means whereon a solid version of the reagent is present. The surface of the holding means may be adapted for holding or immobilizing the reagent. Another example is a reagent providing means 110 that comprises a microfluidic transport means 120 for delivering fluid reagent to the holding means 118 where the reagent can be solidified. The structure of the holding means 118 may be adapted for holding the reagent. The holding means 118 may e.g. comprise an open channel for receiving the reagent in liquid form and for immobilizing the reagent, after solidification and/or drying, in solid version. A reagent overflow chamber 122 may be provided for collecting or discarding excess fluid reagent and an overflow or excess detection mechanism 124 may be provided for controlling the amount of reagent provided onto the holding means. The detection mechanism 124 may assist in controlling appropriate filling of the holding means 118, e.g. with a controlled amount of reagent. The holding means 118 may be adapted for immobilizing the reagent, i.e. it may be an immobilizing means. Exemplary embodiments will be described in more detail below.

The reagent, introduced into the detection chamber 102 using the reagent providing means 110 is preferably a dissolvable reagent, i.e. a reagent adapted for dissolving when in contact with the fluid sample. The reagent may be assisting in label-based analyte detection. It may comprise reagents of chemical or biochemical nature for reacting with the analyte to produce a detectable signal that represents the presence of the analyte in the sample. For instance, the reagent may comprise a probe or a labeled probe. In a particular embodiment, the reagent comprises probes labeled with magnetic or magnetisable particles. Suitable reagents for use in different detection systems and methods include a variety of active components selected to determine the presence and/or concentration of various analytes. There are numerous chemistries available for use with each various analytes. They are selected with respect to the analyte to be assessed. In one example, the probe comprised in the reagent is an antibody. In other examples, the reagent may contain for example an enzyme, a co-enzyme, an enzyme inhibitor, an enzyme substrate, a co-factor such as ATP, NADH, etc. . . . to facilitate enzymatic conversion, a vitamin, a mineral, the invention clearly not being limited thereto. For example, the reagent can include one or more enzymes, co-enzymes, and co-factors, which can be selected to determine the presence of metabolites or small molecules in a sample. Furthermore, the reagent may also comprise labels, buffer salts, detergents, sugars, etc. . . . Multiple different reagents may be present in separate structures to enable assays with different labels or under different conditions, driven by solution composition.

The solid version of the reagent 112 may be a dried or lyophilized form. This results in a long shelf life, i.e., good properties during storing whereby, e.g. interaction prior to addition of fluid sample is limited. In one particular embodiment, the reagent is comprised in a porous material, e.g. it forms a porous layer. The latter may be obtained by depositing a reagent layer comprising material that sublimes during drying and by drying the reagent layer, e.g., sublimation of water and/or of a salt such as ammonium carbonate. The porous reagent layer thus obtained furthermore may be nano-porous or micro-porous. Porosity is advantageous as it assists in improving the dissolution of the reagent components. The reagent may be held in a cross-linkable polymeric material. The reagent is then immobilized in the holding means by initiating cross-linking of the polymer. In another particular embodiment, the reagent is comprised in one or more soluble lyophilized beads. These beads can be formed, for example, by dropping a solution containing the constituents of the reagent in a freezing medium, followed by freeze-drying of the obtained beads. The reagent may be applied by any suitable micro-deposition technique such as spotting, pipetting, printing, e.g., ink-jet printing at the appropriate position in the microfluidic sensor arrangement, as will be described in more detail below. One alternative is applying the reagents by providing them in a channel in the holding means in liquid form and solidifying the reagents on the holding means, e.g. by natural drying, forced drying or freeze-drying. In case forced drying is applied, any drying device appropriate to obtain a solid version of the reagent is encompassed by the present disclosure, e.g., a (vacuum) oven, a freeze-drier. In still another embodiment, more than one reagent layer can be deposited on top of each other and/or on different substrate surfaces in the sensor arrangement for use in detecting, e.g., beside each other. The site at which the reagent is held is preferably distinct and separate from the sensor surface 104 in some embodiments of the present invention.

As an optional feature, the sensor arrangements of the present invention may further comprise a sample outlet 226 for removing the sample fluid from the detection region, wherein said sample outlet 226 is distinct from the sample inlet 108 in which the fluid sample is admitted and also distinct from the reagent providing means, i.e. the reagent inlet for introducing the reagent, through which the reagent can be introduced into the detection region, via a microfluidic transport means and it can also be distinct from the reagent outlet, if present. In case the device is a reactor, e.g. bioreactor, the product may be collected through the sample outlet 226.

As described above, the sensor surface 104 may be part of a sensor 116 or cooperate with an external sensor. The detection sensor 116 may include any suitable sensor, e.g., a magnetic, mechanical or optical sensor, although the invention is not limited thereto. The magnetic sensor may for example be a Hall sensor or may include a magneto-resistive element such as a GMR, TMR or AMR sensor. Further, an excitation means 128 may be provided, for example, a source of light for exciting labels assisting in the detection or a magnetic field for, e.g., activating magnetic beads carrying the reagent. The sensor arrangement may further comprise a processing means 130 for processing the sensor results thus allowing the provision of a suitable output. Such processing means 130 may be any suitable means such as for example a computing means. As an optional feature, the sensor arrangement may further comprise retention means 132 for retaining the reagent or components thereof on the holding means. Such retention means allows both holding the reagent or components thereof and releasing the reagent or components thereof if a different timing than that obtained by natural dissolution and diffusion is to be obtained. As an optional feature, according to some embodiments of the present invention, the microfluidic sensor arrangement may further comprise actuation means 134. The actuation means 134 may be mixing means and/or may be means for positioning or displacing components of the fluid mixture, e.g., after contacting the sample fluid with the reagent.

Similarly, as an optional feature, according to some embodiments of the present invention, the sensor arrangement may further comprise temperature control means 136. The temperature control means 136 may control or change the temperature within the detection chamber 102 in order to optimize the interactions between the sample fluid and the reagent. These temperature control means may comprise a heating, e.g., electric resistance and/or a cooling element, e.g., a Peltier cooler. Preferably, the temperature control means are situated below and/or above the sensor surface in order to affect the temperature of the detection chamber. The temperature control means 136 may also be located outside of the detection region in the detection chamber, in order to control the course and/or the rate of (bio)chemical reactions or specific properties of the sample (such as viscosity) that may affect the desired result.

The first aspect of the present invention will now further be described by a number of particular embodiments, the present invention not being limited thereto, but only by the claims.

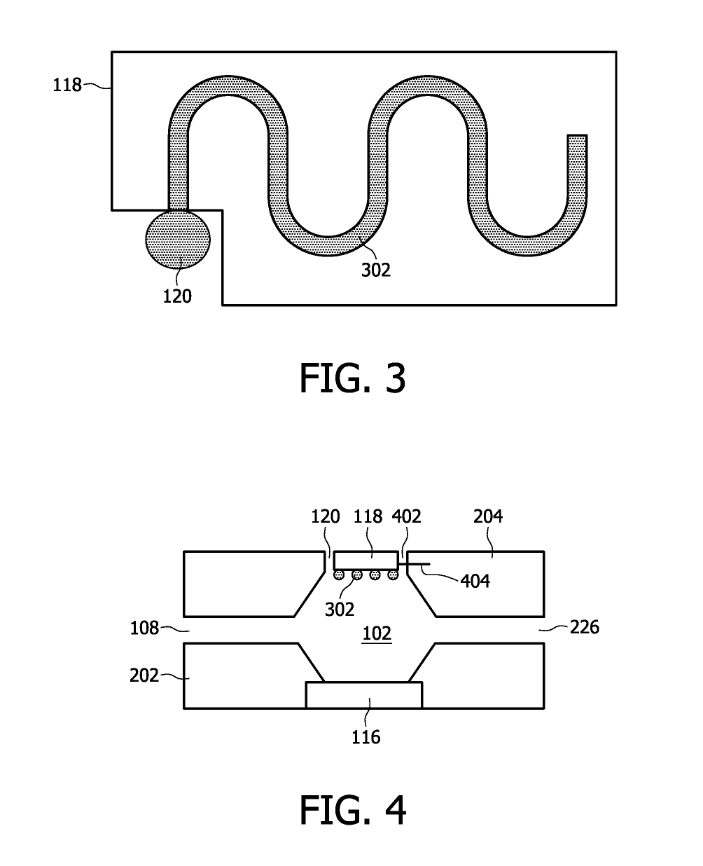

In a first particular embodiment according to the first aspect, the reagent providing means 110 is adapted for introducing the reagent in a liquid form and to deliver it to the holding means 118 where it can be solidified and/or dried. By drying the solvent may be removed from the reagent resulting in a solid reagent. The detection chamber has been completed substantially before this process, e.g. it is already enclosed, in such a way that the sample inlet may be already fully formed and optionally also already been treated, prior to providing the reagent. The reagent providing means therefore comprises a microfluidic transport means 120, connected to a holding means 118 within the detection chamber 102. The holding means may determine a reagent region within the detection chamber 102 where the reagent is held. The shape or nature of the holding means may also determine the quantity of reagent held within the detection chamber. The holding means thereby is located at a selected surface within the detection chamber 102 so that the reagent comes into fluid contact with the sensing surface when the fluid sample is introduced into the detection chamber. The distance between the holding means and the sensor surface may be set in order to determine a rate of reaction of the reagent and the sample fluid and its effect upon the sensor surface. More than one reagent can be introduced in the holding means 118, in a sequential manner or as a mixture. Alternatively, in other embodiments of the invention, multiple holding means can be present, with common or own inlets. The microfluidic transport means 120 and/or the holding means 118 may comprise microfluidic structure, e.g. a capillary, e.g., a tube, a hollow channel section, multiple fine channels, or a porous structure consisting of a “wood” of regular pillars or a random structure such as a wicking material or glass fibre pad, with dimensions such that liquid, e.g., a reagent solution, can be driven therein and along via capillary forces. Typical dimension for capillary sections are 0.1 to 2 mm. The sensor arrangement may further comprise pressure means for forcing the reagent through the reagent inlet into the microfluidic transport means connected to the holding means. Suitable pressure means comprise but are not limited to, e.g., pumps, syringes and the likes. Preferably, the capillary is dimensioned in such a way that the reagent does not flow into parts other than the detection chamber and does not flow to other parts than the reagent region. Moreover, its dimensions can be adapted to determine a predetermined amount of reagent contained in the capillary, which will be put in contact with the fluid sample when the latter is introduced into the detection chamber 102. More generally, the holding means 118 may be adapted for holding or immobilizing a predetermined amount of reagent. Additionally, said capillary may be hydrophilic or may be made hydrophilic by a coating in order to accommodate aqueous samples, as will be described below. The reagent providing means 110 may be placed on any suitable place in the outer wall, distinct from the sample inlet 108. For instance, the reagent providing means 110 may delimit the top of the detection region, e.g., detection chamber. Alternatively, the reagent may be situated between the sensing surface and the surface delimiting the opposite side of this region. It is also possible to realize a detection region, e.g., detection chamber having two or more holding means carrying at least one reagent. By way of illustration, FIG. 2 and FIG. 3 illustrate examples of a microfluidic sensor arrangement according to the first aspect. FIG. 2 shows a vertical cross sectional view of such an exemplary sensor arrangement 100. The sensor arrangement 100 comprises a reagent providing means 110 comprising a microfluidic transport means 120. It is to be noted that the microfluidic transport means 120 may be a fluidic structure, e.g., a capillary, more preferably a hydrophilic capillary, and that it is distinct from the sample fluid inlet 108. The microfluidic transport means 120 is connected to a holding means 118, on which the reagent can be held in solid form. The surface of the holding means 118 may be adapted for holding the reagent, e.g. by comprising an open channel, open towards the detection chamber, and comprising capillary properties and/or hydrophilic properties for holding and easily filling of the channel. The length of the channel thereby may be adapted for holding a predetermined amount of reagent to be applied to the holding means 118. An example of a possible shape of such a channel 302 is illustrated in FIG. 3. The amount of reagent that can be stored in the holding means can be determined by the length of the channel, e.g., by the number of meanders in the structure shown in FIG. 3. Similar ways to control the amount of reagent exist for other capillary structures.

In a second particular embodiment, a sensor arrangement as discussed in the first particular embodiment is described, whereby the sensor arrangement 100, further comprise a reagent overflow chamber 402 for the reagent for collecting excess reagent provided to the holding means. The latter is illustrated in FIG. 4. In case the holding means 118 comprises a channel 302 for holding the reagent, the reagent overflow chamber 402 may be positioned at the opposite side of the channel as where the inlet for the channel 302 is provided. In case too much reagent is applied in the holding means 118 through the microfluidic transports means 120, relative to the volume that can be held on the holding means 118, the excess reagent will be evacuated through the overflow chamber 402. Optionally, the overflow chamber may be a chamber located within the device or outside of it. Alternatively, the overflow chamber simply consists of a hole at the end of the holding means, e.g. at the end of the channel in the holding means. In that case, it is envisioned that the channel comes out onto the outside of the device. In one particular embodiment, the overflow chamber itself is a channel. In a further embodiment, the overflow chamber is hydrophilic or made hydrophilic, as described above. In order to control the provision of reagent on the holding means, e.g. in order to avoid overfilling the capillary, the reagent overflow chamber 402 may be equipped with an overflow detection means 404 to detect liquids, e.g., a fluid sensor. This fluid sensor can be used in combination with dosing equipment and/or the design of the holding means to provide a measured amount of reagent within the fluidic structure, e.g., capillary. The fluid sensor can be connected to the dosing equipment, and can give a signal when the reagent reaches the outlet. When the dosing equipment has given the desired amount of reagent, and the reagent has not reached the outlet within a certain time, the fluid sensor will not give a signal. The fluid sensor can be a simple wetting sensor, i.e., two electrodes are sufficient to measure resistance or capacitance at the outlet as is well known by a person skilled in the art. The overflow detection means may be used to verify proper filling of the cartridge with the reagent. The system may include a feedback system providing information about the filling of the holding means. Such feedback may be provided to a dosing system. In one example, the chosen solution for feedback in the dosing procedure is a fluid sensor installed at the outlet of the holding means for the reagent. This sensor thus can be used in combination with dosing equipment. The sensor can be connected to the dosing equipment, and can give a signal when the reagent solution reaches the outlet. When the dosing equipment has given the desired amount, and the reagent solution has not reached the outlet within a certain time, the sensor will not give a signal, indicating to the dosing equipment that further filling is required.

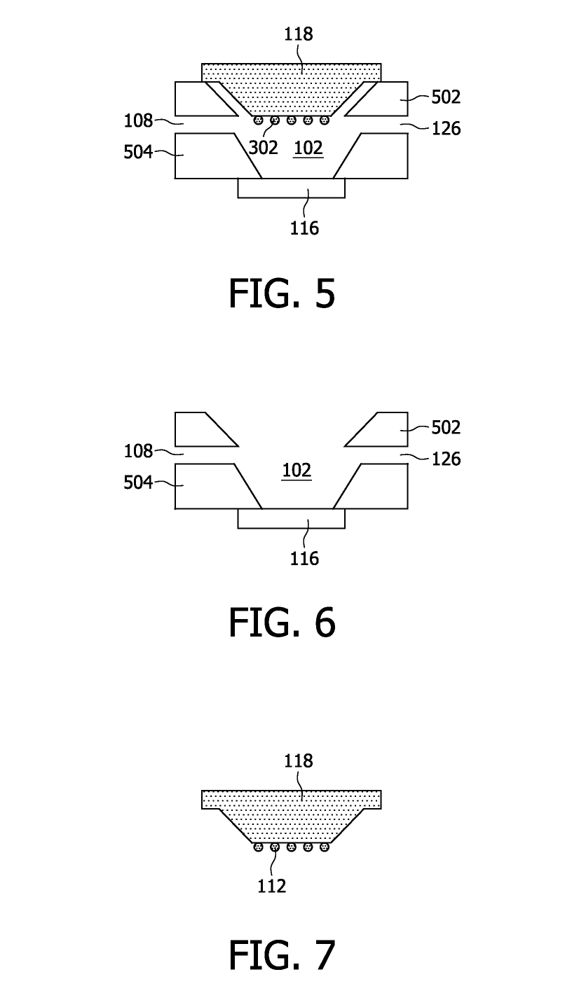

In a third particular embodiment, the detection region, e.g., detection chamber may be formed by an assembly of a sensor-supporting element and a micro fluidic part comprising the sample inlet on the one hand and a holding means being a substrate, which may also be referred to as cover as it covers at least part of the entrance provided by the reagent providing means, and comprising the reagent on another hand. The reagent thus may be applied to the surface of a substrate, wherein said substrate is adapted to fit in the reagent inlet of the detection chamber, which is distinct from the fluid sample inlet. More than one reagent may be applied simultaneously or sequentially on the substrate and/or more than one substrate may be used simultaneously or sequentially in the detection process. According to these embodiments, the substrate comprises the reagent in such a way as to make said reagent accessible to the sample fluid when the substrate is fitted on the reagent inlet of the detection chamber. The holding means may be fixed to the outer wall of the detection chamber in any suitable way, e.g. by gluing, clipping, clicking, screwing etc. The substrate thus may act as a lid forming a side top or wall, e.g. roof, of the detection region. The latter allows separate manufacturing of a component for the device comprising the lid and a component for the device comprising the sensor surface and at least the sample inlet but optionally also a sample outlet. This therefore allows independent manufacturing, thus resulting in independent degrees of freedom for manufacturing these components. By way of illustration, the present invention and the preferred embodiment, not being limited thereto, an example of such an embodiment is shown in FIG. 5 to FIG. 7. FIG. 5 shows a component of the device comprising the holding means 118, e.g., as a lid, from a vertical cross section. The holding means 118 comprises a reagent applied on a central portion thereon. FIG. 6 shows, in vertical cross section view, the same device without the holding means 118 and comprising the sensor 116 with sensor surface, on the bottom part, and a reagent provision means comprising a reagent inlet where the holding means 118 fits. FIG. 7 shows, in vertical cross sectional view, the holding means 118 carrying the reagent in solid version 112. To fix the holding means 118, e.g. lid, in the reagent inlet of the device, use can be made, for example, of an adhesive, clipping means, clicking means, screwing means, etc.

A further advantage of the invention is that the sensor arrangements of the invention advantageously provide for the optimization of the control of the interactions between the fluid sample and the reagent. Indeed, the distance between the reagent and the sensing surface may be selected such that at least a minimal interaction or mixing time occurs before the components of the fluid sample interacted with the reagent reach the sensor surface. In this way, the interaction or mixing time between the fluid sample and the reagent may be selected or tuned. An aspect of the present invention is to provide a distance between the reagent and the sensing surface such that an interaction time of at least 1 second and preferably an interaction time in the range of 5 to 60 seconds is provided. This time can be tuned, e.g., by changing the distance reagent-sensor or, in case magnetic means are employed, by changing the magnetic force for a given distance.

The different elements of the microfluidic sensor arrangement may be organised in various ways. For instance, in a particular embodiment, the reagent providing means is comprised in a first body 202, while the sensor surface 104 is comprised in a second body 204, wherein the first and second bodies are assembled to form a sensor arrangement for use in detecting the presence of an analyte in a fluid sample, as indicated in FIG. 2 and FIG. 4. In another embodiment, the first body 202 further comprises an overflow chamber located inside or outside of the body and a holding means, e.g., capillary, coupling the reagent providing means 110 to the overflow chamber located near the holding means 118. In yet another embodiment, the device comprises only one body in which all the necessary and optionally also optional elements as described above are introduced.

In a second aspect, the present invention relates to a process for manufacturing a microfluidic sensor arrangement for use in detecting the presence of an analyte in a sample fluid. The device may be a device as described in the first aspect of the present invention, comprising the same features and advantages. The manufacturing process comprises providing a sensor surface and providing a housing enclosing the sensor surface and forming the detection chamber. Providing a housing thereby comprises providing a housing with a sample inlet and at least one reagent providing means, distinct from the sample inlet and suitable for introducing at least one reagent into the detection chamber for providing the reagent on at least one holding means distinct from the sensing surface and adapted for holding a solid version of the at least one reagent at a reagent region within the detection chamber. The holding means thereby can be positioned on a selected surface within the detection chamber so that the reagent held by the holding means comes into fluid contact with the sensing surface when the fluid sample is introduced in the detection chamber. Providing a housing may comprise assembling different components such that the detection chamber and the sample inlet is formed. It is an advantage of such a manufacturing technique that a reagent providing means is provided allowing loading of the detection chamber with reagent after assembly of the majority of components, i.e. after assembly of the sensing surface, sample inlet, housing and optionally the sample outlet. The latter is advantageous as it allows late functionalising of the sensor arrangement and/or treatment of different components of the sensor arrangement prior to the provision of the reagent.

As described above, the process of this second aspect comprises providing a sensing surface. The sensing surface 6 may be obtained pre-made whereon biologically or biochemically active moieties are already provided, or it may be obtained via the coating of a sensor or sensing surface with biologically or biochemically active moieties. The process of this second aspect further comprises forming a detection region delimited at its bottom by the sensing surface 6 and at its upper part, opposite the sensing surface, by a substrate or one or more openings, e.g., forming a detection chamber 102 comprising the sensing surface 104 and an upper part, opposite the sensing surface.

The detection chamber of the microfluidic sensor arrangement of the invention may be manufactured through various techniques known in the art, e.g., extrusion-moulding, moulded interconnect devices (MID), press-moulding, injection moulding, (hot) embossing, casting (PDMS), lithography (SU8), (wet) etching (glass). The various components of the device (micro fluidic transport means, holding means, sensing surface . . . ) are then positioned within and around the detection chamber so formed and fixed in any suitable way, e.g., by gluing, clipping, clicking, welding etc. . . . Further assembly of the sensor arrangement for use in detecting also may be performed, i.e., for example providing a detection means, providing a connection means for connecting the detection means to the device in order to obtain a read-out of the detection means used. The present invention advantageously enables the functionalisation/customisation of the device by applying the reagent on the substrate or the fluidic structures, e.g., micro fluidic transport means or holding means, e.g., capillary of the invention to be performed after the manufacturing of the detection, including the creation and optionally the hydrophilising of the sample inlet has been performed but before the device is to be used in a detection analysis. The process of this second aspect of the present invention further comprises providing an inlet and/or an outlet for fluid sample at a location distinct from the reagent inlet. Those inlets and outlets can be formed by any way known to the person skilled in the art such as drilling, boring, punching, cutting, inserting an object, e.g., a hollow tube, and the likes in the detection chamber.

Embodiments of the present invention thus advantageously provide the possibility for hydrophilising the sample inlet and/or other components of the detection chamber prior to the introduction of reagent and e.g. after assembly, allowing to use a hydrophilising fluid, e.g. on the assembled device. The latter assists in an improved manufacturing efficiency. Furthermore, the system is manufactured such that the reagent can be introduced at the end of the manufacturing process, resulting in late functionalising.

As for microfluidic structures such as capillaries, found in, e.g., the microfluidic transport means and the holding means, they usually are made from a polymer, optionally a flexible polymer, e.g., reticulated rubber from a silicon rubber. Such preferred silicon rubbers are polydimethylsiloxanes (PDMS) because of their easy manufacture, gas permeability, inertia and biocompatibility. Additionally, PDMS is easily mouldable and allows reliable production of microfluidic structures at the micro- and even nano-scale. Moreover, the transparency to light and the absence of spontaneous fluorescence of PDMS permits the use of several detection methods in conjunction with these microfluidic structures. However, PDMS is extremely hydrophobic in nature and it is therefore necessary to treat the micro fluidic structures with wetting agents before using them with aqueous samples. Treatments by, e.g., cold oxygen or argon plasmas, adsorbing surfactant, hydrophilic polymers such as Tween 20, Tween 80, Pluronics F80 and the like, are necessary to confer hydrophilic properties to the polymers (see, e.g., EP1750789). Other polymers suitable to make the microfluidic structures of the invention include, but are not limited to, acrylate (PMMA), cyclic olephins (COC), polystyrene (PS), polycarbonate (PC), polyethylene, polypropylene, and polyether imide. Techniques such as, e.g., covalent coupling of hydrophilic materials such as, e.g., PEG, PVA/PVAc, PEI can be used to confer hydrophilic properties to these polymers. The capillary so manufactured is then attached to a body of the sensor arrangement by known means, e.g., gluing, clamping and the likes. Alternatively, such capillary structures can be directly created on a device of the invention through, e.g., etching, carving, melting and the likes. If need be, any or all parts of the device can be flushed with a hydrophilisation solution before or after assembly but prior to applying any aqueous solutions, e.g., fluid sample and/or reagent. The suitable hydrophilising agents comprise all known types of emulsifiers, although polymer hydrophilisation agents with amine groups, amide groups, carboxyl groups and/or hydroxyl groups are preferred. Very good results are achieved particularly with polyvinyl alcohol having a solution viscosity (4% at 20° C. in water) between 4 and 70 mPa·s and a saponification degree of from 80 to 99.5% (see, e.g., U.S. Pat. No. 4,013,617). Typically, the assembled device is flushed with the hydrophilisation solution through the sample inlet, while the reagent is introduced through the reagent inlet connected to a microfluidic transport means, distinct from the sample inlet.

As an optional feature, the distance between the reagent region and the sensing surface may be tuned during manufacturing. This distance should be such as to provide enough time for a proper dissolution of the reagent by the fluid sample and for a proper homogenization of the resulting fluid mixture and to provide for rapid detection. A compromise must therefore be found.

As another optional feature, the process of this second embodiment further comprises providing magnetic actuation means below and/or above the sensor surface. Such actuation means may be embedded in a component, or may be positioned as separate component. It may be performed as part of the assembly of the detection chamber or it may be provided after assembly of the detection chamber.

In a third aspect, the present invention relates to a method for functionalizing at least one microfluidic sensor arrangement, e.g. a microfluidic sensor arrangement as described in any of the embodiments according to the first aspect of the present invention. It thereby is an advantage that this functionalizing can be performed at a late stage in the manufacturing of the microfluidic sensor arrangement, resulting in the possibility to separate the manufacturing of the micro fluidic sensor arrangement completely from the functionalizing of the sensor arrangement. Furthermore it allows to perform treatment of different components such as the sample inlet prior to the introduction of reagent, e.g. dissolvable reagent in the detection chamber. The method comprises introducing a predetermined amount of at least one reagent into the detection chamber. The detection chamber thereby is enclosed within an outer wall comprising a sample inlet and a reagent providing means. The reagent providing means thereby is distinct from the sample inlet. The reagent may be selected from a plurality of reagents, taking into account the application for which the sensor arrangement will be used. Introducing the reagent thereby allows providing the reagent on at least one holding means distinct from the sensing surface in the detection chamber. The method furthermore comprises holding on the at least one holding means a solid version of the predetermined amount of the at least one reagent at a reagent region within the detection chamber at a selected surface within the detection chamber. The reagent thereby is positioned such that the reagent comes into fluid contact with the sensing surface when the fluid sample is introduced in the detection chamber. Introducing the reagent may comprise introducing fluid reagent in a microfluidic structure, e.g. capillary, guiding the reagent to the holding means. Alternatively, the predetermined amount of reagent may be provided in fixed version on a holding means that can be connected to the outer wall of the sensor arrangement. Connecting the holding means to the outer wall of the sensor arrangement then provides the appropriate position of the reagent in the detection region. The reagent may be deposited in any suitable way, such as, but not limited to, e.g., micro-deposition techniques. One example of deposition is dosing, whereby valves are used to control application of small volumes on the central portion of the holding means or in the fluidic structures such as a microfluidic transport means which is adapted for transporting the reagent to the holding means, e.g., via capillary forces. In one embodiment, the reagent thus is provided on the holding means when the holding means is positioned in the detection chamber, by providing a fluid reagent in a microfluidic transportation means in connection with the holding means and introducing the fluid reagent on the holding means. Other techniques may comprise non-contact printing techniques such as inkjet printing or jetting, or contact printing such as tampon printing, micro contact printing, screen printing, stamp printing, etc. . . . The reagent may for instance be deposited as one or more layers. In some embodiments according to the present invention, the method for functionalizing furthermore comprises controlling the amount of reagent provided on the holding means by measuring or detecting an excess of reagent collected in a reagent overflow chamber in connection with the holding means. The latter allows controlling the provision of reagent on the holding means. Both proper filling of the holding means as well as overflow can be determined.

As an optional feature, the reagent may be dried on the surface of the holding means. Drying of the reagent may be performed by application of a low ambient vapor pressure, although the latter is not obligatory. Drying may comprise both drying a reagent from its fluid phase as well as drying a reagent that is already in a solid form after removal of most of the solvent. It may comprise reducing the amount of aqueous components present in the reagent. Heat may be used during drying to improve its efficiency. For instance, the surface of the holding means may be heated. A good drying improves shelf life, i.e., storage properties. In an exemplary embodiment, the ambient atmosphere provided during depositing and/or drying of the reagent has a very low humidity. The latter has the advantage that the drying occurs rapidly. An inert gas can be used in the ambient atmosphere. With very low humidity there is meant a relative humidity less than 30%, more preferably a relative humidity less than 10% and even more preferably a relative humidity of less than 3%. As an optional feature, the reagent may be in a lyophilized form, i.e., has been freeze-dried by first freezing it and afterwards subliming the frozen water formed therein. In other words, a step of lyophilizing also may be applied. Alternatively, the reagent may be provided as associated with a water-soluble polymer, e.g., polyester amide (PEA), polyester urethane (PEUR), or polyester urea (PEU) polymers (see, e.g., WO/2006/083874), which will release the reagent upon contact with the fluid sample. The water-soluble polymers may be manufactured to carry one or more reagent. Yet another alternative is the provision of the reagent as comprised in one or more soluble lyophilized beads. These beads can be formed, for example, by dropping a solution containing the constituents of the reagent in a freezing medium, followed by freeze-drying of the obtained beads as described above.

In a fourth aspect, the present invention, relates to a method for use in detecting the presence of an analyte in a fluid sample. The method preferably may be performed using a microfluidic sensor arrangement as described in the first aspect, although the invention is not limited thereto. The method for use in detecting comprises introducing, via a sample inlet and based on hydrophilic forces, a fluid sample into a microfluidic sensor arrangement. Introducing the sample thus may be performed based on a pulling force exerted by the sample inlet, as the sample inlet is made hydrophilic. The latter allows for an autonomous filling. It allows automatic and/or automated filling of the detection chamber. The microfluidic sensor arrangement thereby may comprise a detection chamber comprising a sensing surface and a reagent in solid form. The method furthermore comprises contacting the fluid sample with the predetermined amount of reagent, thereby forming a fluid mixture. The reagent thereby is accessible to the fluid sample from within the detection chamber. The method furthermore comprises contacting the fluid mixtures with the sensing surface and detecting an interaction between the fluid mixture and the sensing surface. Contacting the fluid sample with reagent may comprise contacting the reagent held or immobilized on a holding means, which may be in a fluidic structure such as a channel in the holding means. In this way, analytes present in the sample fluid may interact with the reagent 7, thus assisting in the detectability of the particles of interest. This contacting step may comprise dissolving a dissolvable matrix wherein reagent components are positioned, e.g., dissolving a reagent layer applied to the holding means. Once the reagent is contacted with the sample fluid, e.g., lyophilized beads of reagent, when used, dissolve and liberate their content. Thereafter, the so formed fluid mixture is contacted with the sensor and wets its surface. The method thus furthermore comprises contacting the fluid mixture with a sensor surface, the sensor surface being distinct from the substrate or fluidic structure and delimiting the detection region. In this way interaction between the particles of interest and the sensor surface is obtained. Such an interaction can be performed rapidly as the sensor surface is initially substantially free of reagent, thus resulting in free areas of interaction for the particles of interest. The detection region may be a detection chamber comprising the holding means and the sensor surface. Furthermore, as the reagent is provided in the detection region, provision of the reagent sufficiently close to the sensor surface assists in a rapid interaction. The method furthermore comprises detecting the interaction between the fluid mixture and the sensor surface. The latter allows to obtain a quantitative or qualitative analysis of the fluid sample, e.g., to obtain information about the presence and quantity of certain components in the fluid sample. The detection of the interaction of the fluid mixture and the sensor surface may comprise the detection of the analyte via detection of specific probes. The probes (e.g., labeled antibodies) and the sensor are both exposed to the analyte and the analyte influences the binding of the probes to the sensor surface. Depending on the type of assays being performed, an analyte labeled with, e.g., a magnetic or magnetisable particle (via a probe) either binds to immobilized capture probes (sandwich assay), or competes with analyte analogues for the binding to immobilized capture probes (competitive assay). After removal of excess (unbound) labeled analytes (which in some embodiments is equivalent with the removal of the magnetic or magnetisable particles), the amount of bound labeled analytes (e.g., labeled with magnetic particles) can be measured. Thus, binding assays may involve adherence of magnetically labeled molecules to the sensor in numbers that reflect the concentration or presence of the analyte molecule. Such tests may, e.g., be used for detecting drugs of abuse, although the invention is not limited thereto. A large number of variations on binding assay methodologies have been described and are all within the scope of the present invention. Detection of a magnetic or magnetisable particle when used as a label is generally done by application of an electric, magnetic, or electromagnetic field and using a magnetic or non-magnetic, e.g., optical or acoustic sensor. Examples of embodiments for the detection of a magnetic or magnetisable particle are given in patent application WO2005/116661 and in references cited therein. Acoustic and/or sonic detection of labels may also be used. In some embodiments, the magnetic particles are only present in the lyophilized beads to enable their manipulation via magnetic means, i.e., magnetic actuation and do not serve as labels. In those embodiments, the detection of the probes on or in the sensor will be adapted to the type of label linked to the probes. Also, the various types of binding and releasing assays may use magnetic particles that comprise optical properties such as, e.g., fluorescent, chromogenic, scattering, absorbing, refracting, reflecting, SE(R)RS-active or (bio)chemiluminescent labels, molecular beacons, radioactive labels, or enzymatic labels. Optically active labels may emit light detectable by a detector, e.g., in the visual, infrared or ultraviolet wavelength region. Nevertheless, the invention is not limited thereto and optical labels, in the present application, may refer to labels emitting in any suitable and detectable wavelength region of the electromagnetic spectrum. According to an embodiment of the third aspect, the present invention also relates to the use of a microfluidic sensor arrangement as described in embodiments of the first aspect for use in detecting an analyte in a fluid sample.

By way of illustration, the present invention not being limited thereto, an example of detection according to the present invention is provided here below and different stages of the manufacturing process are discussed.

The example discusses detection of drugs of abuse (opiates) using a microfluidic sensor arrangement. The principle of detection of drugs of abuse is in the present example based on a magnetic biosensor, whereby bio-chemically functionalized magnetic particles (beads) are used as a marker. These beads bind to a functionalized GMR sensor surface, where they are detected. The GMR sensor is located in a reaction chamber, inside a cartridge that is filled with sample fluids using microfluidic structures. Drug molecules (targets) are detected by a competition/displacement assay, i.e. a biosensor contains a reagent region and a detection region. The reagent contains labels (e.g. magnetic beads) coupled to biologically active moieties (e.g. anti-drug antibodies). The detection region of the sensing surface is provided with a biologically active surface coating (the drug-analogue). When the fluid sample arrives, the reagent dissolves/mixes into/with the sample. Thereafter, or concomitantly, the fluid sample is transported toward the sensing surface and wets the sensing surface. The labeled antibodies as well as the sensing surface are exposed to drug molecules. The free drug molecules influence the binding of labels to the sensing surface, which is detected. Because drug molecules on the surface and in the fluid sample compete with the available antibodies, this assay requires a well-defined number of labeled antibodies. Thus, the detection principle requires that the amount of functionalised magnetic beads in the reaction chamber is well known. The beads are present in dry form in the cartridge and are re-dispersed in the fluid sample as soon as the latter is introduced into the detection chamber.

In the present example, the sample inlet, holding means and microfluidic transport means of the microfluidic sensor arrangement are made hydrophilic, by coating the parts with a hydrophilic material, e.g., a wet treatment with Tween 20. The reagent comprising carboxylated superparamagnetic nanoparticles (iron oxide beads coated with a polymer shell, 500 nm diameter, Adembeads, Ademtech, France) coated covalently with monoclonal anti-morphine antibodies were applied after the hydrophilising, by introducing them via a micro fluidic transportation means and providing a predetermined amount to a holding means as indicated in FIG. 4.

Overload of the holding means of the bead solution in the detection chamber thereby was prevented by an extra hole at the end of a microfluidic structure in the holding means.

The sensing surface was coated with BSA-morphine (Morphine-3-glucuronide) conjugate as the antigen and the binding of the anti-morphine antibody-magnetic particles conjugate to BSA-morphine in the presence of drug-negative or drug-positive fluid samples (in a volume of 1 μl) was detected by reading out the GMR sensor with a specially designed reader.

1. A microfluidic reactor arrangement (100), the reactor arrangement (100) comprising a housing having an outer wall enclosing a reaction chamber (102), the reaction chamber (102) having an interaction surface (104), the outer wall having:
a) at least one sample inlet (108) for introduction of the fluid sample (106), and
b) at least one reagent providing means (110) distinct from the sample inlet (108) for introducing at least one reagent into the reaction chamber (102) thus providing said reagent on at least one holding means (118) for holding a solid version of the at least one reagent at a reagent region within the reaction chamber (102), said holding means (118) being located or locatable on a selected surface distinct from the interaction surface within the reaction chamber (102) so that the reagent held by the holding means (118) comes into fluid contact with the interaction surface (104) when the fluid sample (106) is introduced into the reaction chamber (102).

2. A microfluidic reactor arrangement (100) according to claim 1, the microcluidic reactor arrangement (100) being a microfluidic sensor arrangement (100) for use in detecting an analyte in a fluid sample (106), wherein the reaction chamber is a detection chamber (102) and the interaction surface (104) is a sensing surface (104).

3. A microfluidic reactor arrangement (100) according to claim 1, wherein the reagent providing means (110) comprises a microfluidic transport means (120) for delivering fluid reagent to the at least one holding means (118).

4. A microfluidic reactor arrangement (100) according to claim 1, wherein the holding means (118) is a separate cover connectable to the outer wall of the microfluidic reactor arrangement (100).

5. The reactor arrangement (100) according to claim 1 wherein said holding means (118) is adapted for comprising a predetermined amount of reagent.

6. The reactor arrangement (100) according to claim 1 wherein said holding means (118) comprises an open capillary channel (302).

7. The reactor arrangement (100) of claim 6, comprising a plurality of reagent providing means (110), each of said plurality of reagent providing means (110) being adapted for delivering a reagent.

8. The reactor arrangement (100) according to claim 1 wherein said sample inlet (108) is hydrophilic.

9. The reactor arrangement (100) of claim 1, wherein said reagent providing means (110) comprises a capillary.

10. The reactor arrangement (100) of claim 9, further comprising a sample outlet (126) for removing the fluid sample (106) from the reaction chamber (102), said sample outlet (126) being distinct from said sample inlet (106) and said reagent providing means (110).

11. The reactor arrangement (100) of claim 1 wherein said holding means (118) is connected to a reagent overflow chamber (122).

12. The reactor arrangement (100) of claim 1, wherein said reactor arrangement (100) comprises an excess reagent detection means (124) for detecting excess liquid reagent.

13. The microfluidic reactor arrangement (100) of claim 12, further comprising at least one reagent in a solid version in said holding means (118).

14. A microfluidic reactor arrangement (100) for use in detecting an analyte in a fluid sample, the reactor arrangement comprising a housing having an outer wall enclosing a reaction chamber (102),
a) the outer wall having at least one sample inlet (108) covered with a hydrophilic coating, the sample inlet (108) for introduction of the fluid sample (106);
b) the reaction chamber (102) having an interaction surface (104) and the outer wall having at least one holding means (118) comprising a solid version of at least one reagent at a reagent region within the reaction chamber (102), said holding means (118) being located on a selected surface within the reaction chamber (102) so that the solid reagent held by the holding means (118) comes into fluid contact with the interaction surface (104) when the fluid sample (106) is introduced into the reaction chamber (102).

15. A microfluidic reactor arrangement (100) according to claim 14, the microcluidic reactor arrangement (100) being a microfluidic sensor arrangement (100) for use in detecting an analyte in a fluid sample (106), wherein the reaction chamber is a detection chamber (102) and the interaction surface (104) is a sensing surface (104).

16. A microfluidic sensor arrangement (100) according to claim 14 wherein the reactor arrangement (100) comprises a microfluidic transport means (120) separate from the sample inlet (108) for providing reagent to the holding means 118).

17. A microfluidic sensor arrangement (100) according to claim 14, wherein the holding means (118) comprises an open channel (302) for holding the solid reagent.

18. A method for manufacturing a microfluidic reactor arrangement (100), the method comprising the step of:
a) providing an interaction surface (104)
b) providing a housing enclosing the interaction surface (104) and forming a reaction chamber (102),
said providing a housing comprising providing a housing with a sample inlet (108) and at least one reagent providing means (110), distinct from the sample inlet (108), for introducing at least one reagent into the reaction chamber (102) by providing the reagent on at least one holding means (118) distinct from the interaction surface (104) for holding a solid version of at least one reagent at a reagent region within the reaction chamber, the holding means (118) being positioned on a selected surface within the reaction chamber (102) so that the reagent held by the holding means (118) comes into fluid contact with the interaction surface (104) when the fluid sample is introduced in the reaction chamber (102).

19. The method of claim 18, further comprising hydrophilising the sample inlet (108) by introducing a hydrophilisation liquid in the detection chamber (102) through the sample inlet (102) after said providing a housing and prior to introducing reagent in the sensor arrangement (100).

20. The method of claim 18, further comprising providing a reagent overflow chamber (124) connected to said at least one holding means (118).

21. The method of claim 20, further comprising providing excess detection means (124) for detecting excess reagent liquid in said overflow chamber.

22. The method of claim 18, further comprising introducing a predetermined amount of said at least one reagent via a microfluidic transport means (120) into said holding means (118) and obtaining a solid version of said reagent thereon.

23. A method for functionalizing at least one microfluidic reactor arrangement (100), the at least one microfluidic reactor arrangement (100) comprising a reaction chamber (102) enclosed by an outer wall, the outer wall having a sample inlet (108) and a reagent providing means (110), the method comprising
a) introducing a predetermined amount of at least one reagent into the reaction chamber (102) via the reagent providing means (110) distinct from the sample inlet (108) thus providing the reagent on at least one holding means (118) distinct from an interaction surface in the reaction chamber (102) and
b) holding on the at least one holding means (118) a solid version of the predetermined amount of the at least one reagent at a reagent region within the reaction chamber (102) at a selected surface within the reaction chamber (102) so that the reagent held comes into fluid contact with the interaction surface (104) when the fluid sample is introduced in the reaction chamber (102).

24. A method according to claim 23, the method further comprising detecting an excess of said reagent for controlling the amount of reagent provided on the holding means (118).

25. A method according to claim 23, the method comprising, prior to said introducing, selecting a reagent from a plurality of reagents.

26. A method for detecting an analyte in a fluid sample comprising the step of:
introducing, via a sample inlet (108) and based on hydrophilic forces, a fluid sample (106) into a microfluidic sensor arrangement (100), said microfluidic sensor arrangement (100) comprising a detection chamber (102), said detection chamber (102) comprising a sensing surface (104) and a predetermined amount of reagent in a solid form,
the method further comprising:
contacting the fluid sample with said predetermined amount of reagent, thereby forming a fluid mixture, the reagent being accessible to the fluid sample from within the detection chamber (102);
contacting the fluid mixture with said sensing surface (104); and
detecting an interaction between the fluid mixture and the sensing surface (104).

27. Use of a microfluidic reactor arrangement according to claim 1, for detecting an analyte in a fluid sample (106).
